(19) United States
(12) Patent Application Publication
(10) Pub. No.: US 2017/0100091 A1
(43) Pub. Date: Apr. 13, 2017

(54) ULTRASOUND SYSTEM AND METHOD FOR USE WITH A HEAT-AFFECTED REGION

(71) Applicant: General Electric Company, Schenectady, NY (US)

(72) Inventors: Samset Eigil, Horten (NO); Geir Ultveit Haugen, Oslo (NO); Margot Pasternak, Oslo (NO)

(21) Appl. No.: 14/878,462

(22) Filed: Oct. 8, 2015

(51) Int. Cl. A61B 8/08; A61B 8/00

(52) U.S. Cl. CPC A61B 8/08; A61B 8/463; A61B 8/5223; A61B 8/5238; A61B 8/4444; A61B 8/54; A61B 2018/00577

(57) ABSTRACT

A method and system of ultrasound imaging includes acquiring ultrasound channel data for a region-of-interest, determining an estimated size of a heat-affected region, identifying a first subset of the ultrasound channel data, and identifying a second subset of the ultrasound channel data. The method and system includes generating a pilot trace based on the first subset of ultrasound channel data and comparing the second subset of the ultrasound channel data to the pilot trace to determine delay errors for the second subset of the ultrasound channel data. The method and system includes determining that the heat-affected region has experienced a temperature-induced tissue change based on the delay errors and the estimated size of the heat affected region, and presenting information indicating that the heat-affected region has experienced the temperature-induced tissue change.

### FIELD OF THE INVENTION

This disclosure relates generally to an ultrasound imaging system and method for determining that a heat-affected region has experienced a temperature-induced tissue change.

### BACKGROUND OF THE INVENTION

Thermal ablation is clinically used both for cancer treatment and for the treatment of cardiac irregularities, such as a cardiac arrhythmia. Thermal ablation is used to kill cancerous cells during cancer treatment and to create isolation scars in order to disrupt abnormal electrical pathways in order to treat cardiac arrhythmia. Accurate and precise temperature control is important during a thermal ablation procedure. It is important to raise the tissue to a high enough temperature in order to create irreversible tissue damage, such as that which would be required either to kill cancer cells or to create an isolation scar. Not generating a high enough temperature during a thermal ablation procedure may result in an incomplete ablation, potentially leaving viable cancer cells or incomplete electrical isolation. A procedure that leaves viable cancer cells or results in incomplete electrical isolation is undesirable and may require one or more additional follow-up procedures in order achieve the desired clinical outcome.

Achieving too high of a temperature through thermal ablation may also result in negative effects. For example, too high of a temperature may result in collateral damage to nearby organs. Too high of a temperature may cause both short-term and long-term problems. For example, the ablation may result in too much tissue being destroyed. This may damage nearby organs and/or have catastrophic effects such as major bleeding.

For these and other reasons an improved ultrasound imaging system and method for estimating the temperature of tissue and/or determining a position of a heat-affected region is desired.

### BRIEF DESCRIPTION OF THE INVENTION

The above-mentioned shortcomings, disadvantages and problems are addressed herein which will be understood by reading and understanding the following specification.

In an embodiment, a method of ultrasound imaging includes acquiring ultrasound channel data for a region-of-interest, determining an estimated size of a heat-affected region in response to an application of a thermal source within the region-of-interest, identifying a first subset of the ultrasound channel data, and identifying a second subset of the ultrasound channel data that is different than the first subset. The method includes generating a pilot trace based on the first subset of the ultrasound channel data and comparing the second subset of the ultrasound channel data to the pilot trace to determine delay errors for the second subset of the ultrasound channel data. The method includes determining that the heat-affected region has experience a temperature-induced tissue change based on the delay errors and the estimated size of the heat-affected region. The method includes presenting information indicating that the heat-affected region has experienced the temperature-induced tissue change after determining that the heat-affected region has experienced the temperature-induced tissue change.

In an embodiment, an ultrasound imaging system includes a probe, a display device, and a processor in electronic communication with the probe and the display device. The processor is configured to control the probe to acquire ultrasound channel data for a region-of-interest, determine an estimated size of a heat-affected region in response to an application of a thermal source within the region-of-interest, identify a first subset of the ultrasound channel data, identify a second subset of the ultrasound channel data that is different than the first subset, and generate a pilot trace based on the first subset of the ultrasound channel data. The processor is configured to compare the second subset of the ultrasound channel data with the pilot trace to determine delay errors for the second subset of the ultrasound channel data. The processor is configured to determine that the heat-affected region has experienced a temperature-induced tissue change based on the delay errors and the estimated size of the heat-affected region. The processor is configured to present information indicating that the heat-affected region has experienced the temperature-induced tissue change.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a flow chart of a method in accordance with an embodiment;

FIG. 3 is a schematic diagram of a transducer array and a heat-affected region in accordance with an embodiment;

FIG. 4 is a schematic diagram of a heat-affected region in accordance with an embodiment; and

FIG. 5 is a schematic representation of a display screen in accordance with an exemplary embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

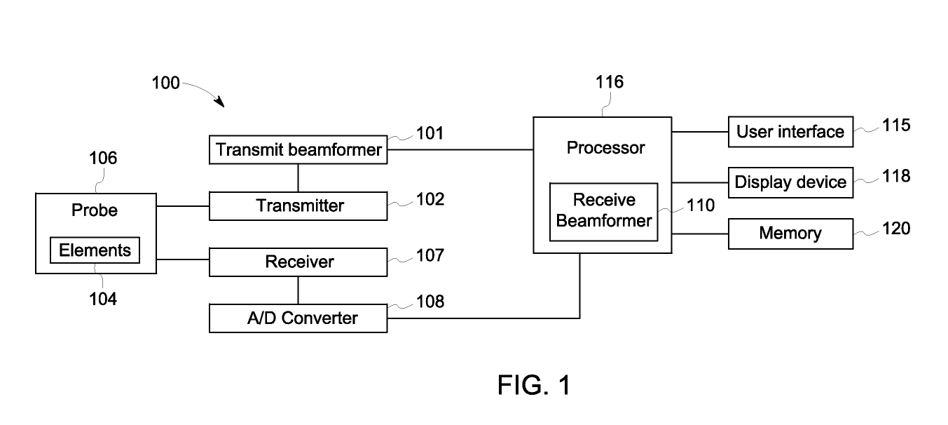

FIG. 1 is a schematic diagram of an ultrasound imaging system 100 in accordance with an embodiment. The ultrasound imaging system 100 includes a transmit beamformer 101 and a transmitter 102 that drive elements 104 within a probe 106 to emit pulsed ultrasonic signals. The probe 106 may be any type of probe, including a linear probe, a curved array probe, a 1.25D array probe, a 1.5D array probe, a 1.75D array probe, or 2D array probe according to various embodiments. The probe 106 may be used to acquire 2D, 3D, or 4D ultrasound data. For 3D and 4D embodiments, each acquired volume may include a plurality of 2D images or slices. Still referring to FIG. 1, the pulsed ultrasonic signals are back-scattered from structures in the body. The echoes are converted into electrical signals, or ultrasound channel data, by the elements 104. The term ultrasound channel data refers to ultrasound data that has not been fully beamformed. Ultrasound channel data may refer to ultrasound data that is completely unbeamformed, partially beamformed, partially delayed or channel data may also refer to partially beamformed ultrasound data that has been summed. The ultrasound channel data is received by a receiver 107. The ultrasound channel data representing the received echoes is passed through an analog to digital (A/D) converter 108, where the ultrasound channel data is converted from analog to digital. The receive beamformer 110 may be a hardware component, such as an Application Specific Integrated Circuit (ASIC), a firmware component such as a field-programmable gate array (FPGA) or a software beamformer. According to some embodiments, the probe 106 may contain electronic circuitry to do all or part of the transmit beamforming and/or the receive beamforming. For example, all or part of the transmit beamformer 101, the transmitter 102, the receiver 107, the A/D converter 108, and the receive beamformer 110 may be situated within the probe 106 in other embodiments. For yet another embodiment the beamforming can use phase delay or sampled delay lines (non-digital) for a pre-beamforming step. For embodiments where the receive beamformer 110 is a software beamformer the receive beamformer 110 may use executable code in order to apply the appropriate delays and sum the IQ data. FIG. 1 shows an exemplary embodiment where the receive beamformer 110 may be a software beamformer. The receive beamformer 110 is depicted as a subcomponent of a processor 116. The receive beamformer 110 may be a separate module within the processor 116, or the function of the receive beamformer 110 may be performed by the processor 116. The receive beamformer 110 applies delays to the ultrasound channel data. The receive beamformer 110 may perform a summing operation after applying the delays to the ultrasound channel data. Ultrasound channel data may be used to refer to ultrasound data emerging from one channel (element) or a selected group of elements. The ultrasound channel data may include either analog or digital ultrasound channel data from the elements 104, the receiver 107, or the A/D converter 108.

The terms “scan” or “scanning” may be used in this disclosure to refer to acquiring ultrasound channel data through the process of transmitting and receiving ultrasonic signals. The terms “data” and “ultrasound data” may be used in this disclosure to refer to either one or more datasets acquired with an ultrasound imaging system. A user interface 115 may be used to control operation of the ultrasound imaging system 100. The user interface 115 may be used to control the input of patient data, or to select various modes, operations, and parameters, and the like. The user interface 115 may include a one or more user input devices such as a keyboard, hard keys, a touch pad, a touch screen, a track ball, rotary controls, sliders, soft keys, or any other user input devices.

The processor 116 controls the transmit beamformer 101, the transmitter 102, the receiver 107, the A/D converter 108, and the receive beamformer 110. The transmit beamformer 101 may be controlled by hardware, firmware or software. The transmit beamformer 101 may also be part of the processor 116. For embodiments where the transmit beamformer 101 is a software beamformer, the transmit beamformer 101 may include one or more of the following components: a graphics processing unit (GPU), a microprocessor, a central processing unit (CPU), a digital signal processor (DSP), or any other type of processor capable of performing logical operations. And, as described above, the receive beamformer 110 may be a hardware, firmware or a software beamformer according to various embodiments. For embodiments where the receive beamformer 110 is a software beamformer, the receive beamformer 110 may include one or more of the following components: a graphics processing unit (GPU), a microprocessor, a central processing unit (CPU), a digital signal processor (DSP), or any other type of processor capable of performing logical operations controlled by a software program. The receive beamformer 110 may be configured to perform conventional beamforming techniques as well as techniques such as retrospective transmit beamforming (RTB).

The processor 116 is in electronic communication with the probe 106. The processor 116 may control the probe 106 to acquire ultrasound channel data. The processor 116 controls which of the elements 104 are active and the shape of a beam emitted from the probe 106. The processor 116 controls the transmit beamformer 101 and the transmitter 102 to control a focus of the transmit beams. The processor 116 controls the receiver 107, the A/D converter 108 and the receive beamformer 110 to perform dynamic focusing while receiving ultrasound data. The processor 116 is also in electronic communication with a display device 118, and the processor 116 may control the receive beamformer 110 to apply beamforming to the ultrasound channel data and perform additional processing in order to display images based on the beamformed ultrasound data on the display device 118. For purposes of this disclosure, the term “electronic communication” may include both wired and wireless connections. The processor 116 may include a central processing unit (CPU) according to an embodiment. According to other embodiments, the processor 116 may include other electronic components capable of carrying out processing functions, such as an application specific integrated circuit (ASIC), a digital signal processor (DSP), a field-programmable gate array (FPGA), a graphics processing unit (GPU) or any other type of processor capable of executing logical operations. According to other embodiments, the processor 116 may include multiple electronic components capable of carrying out processing functions. The processor 116 may be adapted to perform one or more processing operations on the ultrasound data according to a plurality of selectable ultrasound modalities. The ultrasound data may be processed in real-time during a scanning session as the ultrasound data is received. For the purposes of this disclosure, the term “real-time” is defined to include a procedure that is performed without any intentional delay. Real-time frame rates or volume rates may vary based on the size of the region or volume from which data is acquired and the specific parameters used during the acquisition. The data may be stored temporarily in a buffer (not shown) during a scanning session and processed in less than real-time in a live or off-line operation. Some embodiments may include multiple processors (not shown) to handle the processing tasks. Or, the processing functions attributed to the processor 116 and the receive beamformer 110 may be allocated in a different manner between any number of separate processing components, including a multi-core processor, or configurations where the processor 116 includes multiple separate processors.

According to an embodiment, the ultrasound imaging system 100 may continuously acquire ultrasound channel data at a frame-rate of, for example, 10 to 100 Hz. Images generated from the ultrasound channel data may be refreshed at a similar frame-rate. Other embodiments may acquire and display data at different rates. For example, embodiments may acquire ultrasound data at a frame rate of less than 10 Hz or greater than 100 Hz depending on the size of the volume and the intended application. A memory 120 is included for storing processed frames of acquired data. In an exemplary embodiment, the memory 120 is of sufficient capacity to store frames of ultrasound data acquired over a period of time at least several seconds in length. The frames of data are stored in a manner to facilitate retrieval thereof according to time of acquisition. The memory 120 may comprise any type of data storage medium.

Optionally, embodiments may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed with mode-related modules by the processor 116 (e.g., B-mode, Color Doppler, M-mode, Color M-mode, spectral Doppler, Elastography, TVI, strain, strain rate, and the like) to form 2D, 3D, or 4D images or data. For example, one or more modules may generate B-mode, color Doppler, M-mode, color M-mode, spectral Doppler, Elastography, TVI, strain, strain rate and combinations thereof, and the like. The image frames are stored and timing information indicating the time of acquisition may be recorded. The modules may include, for example, a scan conversion module to perform scan conversion operations to convert the image frames from coordinates beam space to display space coordinates. A video processor module may be provided that reads the image frames from a memory and displays the image frames in real time while a procedure is being carried out on a patient. A video processor module may store the image frames in an image memory, from which the images are read and displayed.

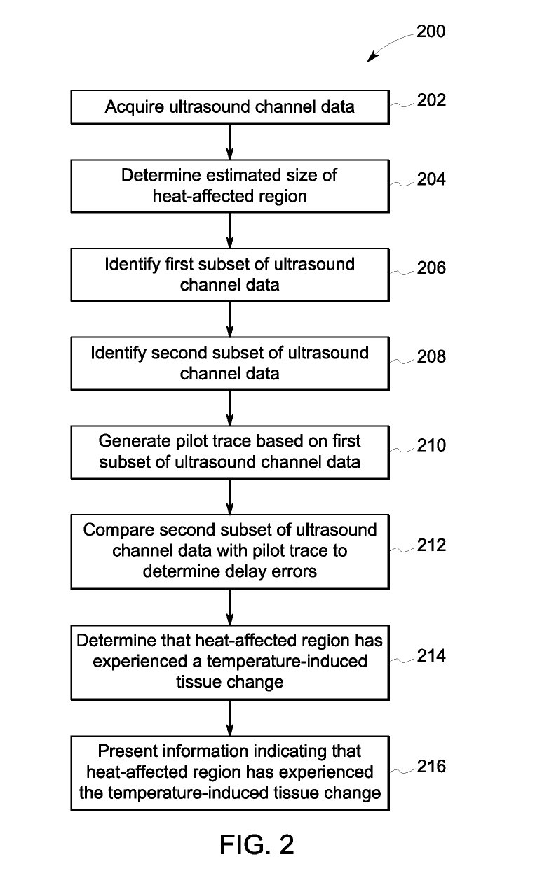

FIG. 2 is a flow chart of a method 200 in accordance with an exemplary embodiment. The individual blocks of the flow chart represent steps that may be performed in accordance with the method 200. Additional embodiments may perform the steps shown in a different sequence and/or additional embodiments may include additional steps not shown in FIG. 2. The technical effect of the method 200 is the presentation of information indicating that the heat-affected region has experienced a temperature-induced tissue change.

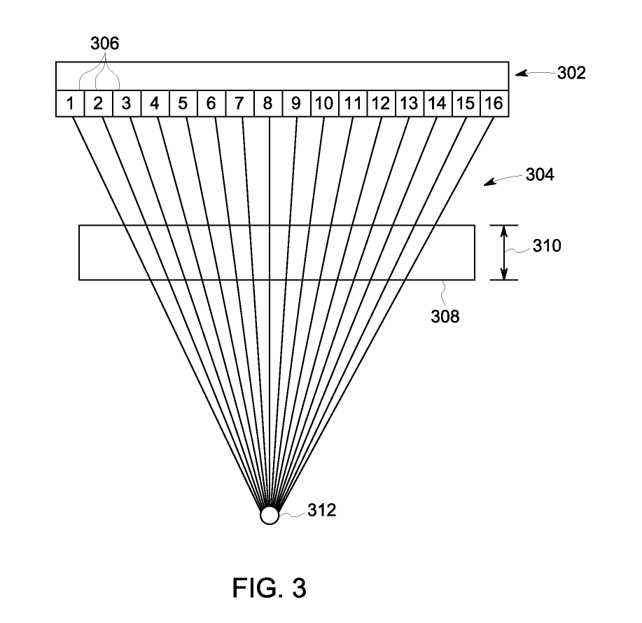

FIG. 3 is a schematic representation of a transducer array 302 and a plurality of ultrasound propagation paths 304 according to an embodiment. FIG. 3 shows an exemplary embodiment where the transducer array 302 includes 16 elements 306. It should be appreciated that other embodiments may have a different number of elements and/or the elements may be arranged in an array of a different configuration. According to an embodiment a single discrete channel may be associated with each of the elements 306. According to other embodiments, signals from multiple different elements may be routed to a common channel through techniques including sub-aperture processing. Each element 306 is labeled with an integer from 1 through 16. For purposes of discussing FIG. 3, the channel associated with a given element 306 will be identified by the same integer as the element. For example, channel 1 will refer to the channel receiving ultrasound data from element 1, channel 2 will refer to the channel receiving ultrasound data from element 2, etc. FIG. 3 also includes a heat-affected region 308 with a thickness 310.

The method 200 will be described by referencing FIGS. 1, 2, and 3. At step 202, the processor 116 controls the probe 106 to acquire ultrasound channel data. As described previously, the processor 116 may control the transmit beamformer 101, the transmitter 102, the receiver 107, the A/D converter 108, and the receive beamformer 110 in order to acquire ultrasound channel data with the elements 104 in the probe 106. As previously described, the term “ultrasound channel data” refers to ultrasound data that is collected by a channel or a selected group of channels. The ultrasound channel data may be acquired by transmitting an ultrasound beam focused on one or more focal points and then dynamically focusing while receiving the ultrasound channel data along a plurality of beams. Ultrasound channel data may also be acquired through a multi-line acquisition process where multiple receive lines are acquired for each transmit event.

According to an exemplary embodiment, a thermal source may be applied to the patient during the process of acquiring the ultrasound channel data during step 202. For example, an ablation catheter may be used to adjust the temperature of tissue within the patient. The ablation catheter may use various techniques to adjust the temperature of the patient's tissue including radio-frequency (RF) ablation, cryoablation, or any other technique of heating or cooling a patient's tissue in a targeted manner. According to an exemplary embodiment, the thermal ablation may be used for various purposes such as annihilating cancerous tissue or performing a cardiac ablation procedure in order to alter electrical pathways across the patient's heart. It should be appreciated that the method 200 may be used in combination with other procedures according to additional embodiments.

At step 204, the processor 116 determines an estimated size of a heat-affected region, such as the heat-affected region 308. The method 200 will be described according to an embodiment where an ablation catheter is used to heat tissue within the patient during the process of acquiring ultrasound channel data at step 202. Those skilled in the art should appreciate that the method 200 may be used with other types of procedures as well.

The processor 116 may determine the estimated size of the heat-affected region 308 through many different techniques such as implementing a bio-heat equation or implementing a heuristic approach. Exemplary embodiments involving implementing a bio-heat equation and implementing a heuristic approach will be described below.

According to an exemplary embodiment, the processor 116 may use a bio-heat equation to determine the estimated size of the heat-affected region 308. The bio-heat equation may, for instance, express a relationship describing how heat spreads in biological tissue in response to the application of a known thermal source for a known amount of time. For example, by inputting a known thermal source and the amount of time that the thermal source is applied; the processor 116 may implement the bio-heat equation to estimate the size of the heat-affected region 308. The size of the heat-affected region 308 may include a thickness of the heat-affected region 308, or it may include a radius or diameter of the heat-affected zone depending upon the embodiment.

The Pennes bio-heat equation may be used according to an embodiment. The Pennes bio-heat equation is shown below:

$$ρ C \frac{d T}{d t} = Δ \cdot k Δ T + Q + {Q}_{B} + A$$

Where ρ is density, C is specific heat, T is temperature, k is thermal conductivity, Q is the microwave power density, Q_{B} accounts for the effects of perfusion and A is the metabolic heat generation term.

According to one exemplary embodiment, it is assumed that the thermal input results in a heat-affected region with uniform thickness. Then, the Pennes bio-heat equation may be implemented by setting both Q_{B} and A to zero. Additionally, the speed of sound may be assumed to vary in a linear manner with temperature. For example, the speed of sound may be assumed to vary approximately 3 m/s/C.° according to an embodiment. Based on the above assumptions (i.e., setting both Q_{B} and A to zero, and assuming the speed of sound varies in a linear manner with temperature), it is possible to implement the Pennes bio-heat equation, or a different bio-heat equation, to determine the estimated size of a heat-affected region 308 in response to the application of a known thermal source for a known amount of time

The processor 116 may also implement a heuristic approach to determine the estimated size of the heat-affected region 308. For example, the processor 116 may access a look-up table in order to determine the estimated size of the heat-affected region 308 in response to the application of a known thermal source for a known amount of time. The look-up table may, for instance, include values correlating the thermal source and the time of application to the estimated size of the heat-affected region 308. The values in the look-up table may be generated from empirical data or the values in the look-up table may be estimated based on a model. The use of a look-up table is just one example of a heuristic approach. It should be appreciated that other embodiments may involve the implementation of heuristic approaches other than a look-up table.

At the conclusion of step 204, the processor 116 has determined an estimated size of the heat-affected region 308. The use of the estimated size of the heat-affected region 308 will be discussed in detail hereinafter.

At step 206, the processor 116 identifies a first subset of the ultrasound channel data. The first subset of the ultrasound channel data may include data from one or more channels acquired with a portion of the elements 104. For example, the first subset of the ultrasound channel data may include ultrasound channel data from a number of channels acquired with a central portion of an array of elements 104. Various embodiments may use a different number of channels in the first subset of ultrasound channel data. Additionally, in some embodiments, the channels in the first subset may be associated with elements that are not adjacent to each other. According to an exemplary embodiment, a probe may include an array with 128 elements. The first subset may, for instance, include the ultrasound channel data associated with 2 central elements, 4 central elements, or any other number of elements that is less than the total number of elements. Additionally, the first subset of the ultrasound channel data may be selected so that the channels are associated with a subset of elements that are not centrally located on the array. For example, the first subset of channels may be offset to one side of the array.

At step 208, the processor 116 identifies a second subset of the ultrasound channel data. According to an embodiment, the second subset of the ultrasound channel data may not overlap with the first subset of ultrasound channel data. According to other embodiments, some of the channels in the first subset of ultrasound channel data may be the same as some of the channels included in the second subset of ultrasound channel data.

At step 210, the processor 116 generates a pilot trace based on the first subset of the ultrasound channel data. If the first subset of the ultrasound channel data includes data from just a single channel, then the pilot trace may include just the data from that single channel. However, for embodiments where the first subset of ultrasound channel data includes a plurality of channels, the processor 116 may generate the pilot trace by averaging the data from the plurality of channels. The pilot trace may be generated by calculating an arithmetic mean, a weighted average, or any other technique based on the first subset of the channel data. The processor 116 may also apply one or more smoothing techniques during the process of generating the pilot trace from the first subset of the channel data. Using multiple different channels to generate the pilot trace may result in a more robust estimate of the delay time associated with the pilot trace since averaging multiple channels minimizes the effects of any noise present in any single channel.

At step 212, the processor compares the second subset of ultrasound channel data with the pilot trace to determine a delay error for each of the channels in the second subset of the ultrasound channel data.

According to an exemplary embodiment, the first subset of the ultrasound channel data may include the two center channels, i.e., channel 8 and channel 9, and the second subset of the ultrasound channel data may include the remaining 14 channels. In other words, the second subset of the ultrasound channel data may include channels 1, 2, 3, 4, 5, 6, 7, 10, 11, 12, 13, 14, 15, and 16. The processor 116 may therefore generate the pilot trace from channel 8 and channel 9. According to an embodiment, the processor 116 may average channel 8 with channel 9 to generate the pilot trace.

As previously discussed, during step 212, the processor 116 may compare the second subset of ultrasound channel data (i.e., channels 1, 2, 3, 4, 5, 6, 7, 10, 11, 12, 13, 14, 15, and 16) with the pilot trace to determine a delay error for each of the channels. FIG. 3 shows an exemplary embodiment where the face of the transducer array 302 is generally parallel to the heat-affected region 308 using 16 channels to illustrate the concept. In most cases the channel count will be much higher. While this may be an advantageous configuration for reasons that will be discussed below, it should be appreciated that the technique may be used with the probe positioned differently with respect to the heat-affected region 308 according to other embodiments. Additionally, the heat-affected region 308 may be shaped differently in other embodiments. However, an exemplary embodiment will be described with respect to the orientation depicted in FIG. 3.

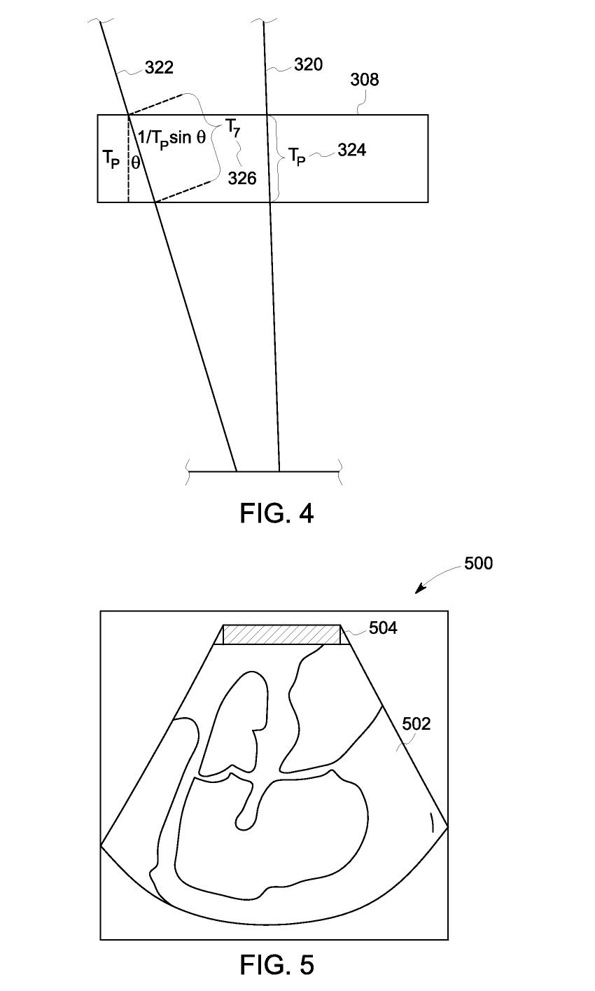

FIG. 4 is a schematic representation of a zoomed-in view of a portion of the heat-affected zone 308 shown in FIG. 3. A pilot trace 320 and an ultrasound beam 322 are both shown with respect to the heat-affected zone 308. The signal representing the ultrasound beam 322 may be acquired by channel 7 according to an embodiment. The pilot trace 320 is positioned between elements 8 and 9 since the pilot trace 320 is an average of the ultrasound channel data acquired from element 8 and element 9.

According to the geometry shown in FIG. 3 and FIG. 4, the pilot trace 320 represents an average transit time for a pulse that travels through the heat-affected region 308. According to the embodiment shown in FIGS. 3 and 4, the pilot trace represents the shortest transit time for a pulse that travels through the heat-affected region 308. Since the pilot trace 320 is generated based on channel data associated with the center elements and because the array is generally parallel to the heat-affected region 308, the pilot trace 320 represents the shortest distance between reflector 312 and the array 302. It is not necessary that the pilot trace 320 is exactly perpendicular to the heat-affected region 308. In other embodiments, the pilot trace may pass through the heat-affected region 308 at an angle. Those skilled in the art will appreciate that other embodiments may have different geometries between the array 302 and the heat-affected region 308. The processor 116 is able to calculate the expected delays for each of the channels based on the pilot trace 320. For example, the processor 116 can determine the relative path length differences between the beams for each of the channels and pilot trace 320 based on the geometry of the transducer array 302, the elements used to collect the ultrasound data for each specific channel, and the position of the focal point of the transmitted beams. The processor 116 is therefore able to calculate the expected delay for each of the other channels. Since the pilot trace 320 is an average time through the heat-affected region 308 based on multiple beams, the delay associated with the pilot trace 320 incorporates the change in the speed of sound through the heat-affected region. However, channel data acquired from beams that pass through the heat-affected region 308 at an angle will show a delay that is offset from the expected delay because the beam travels through more of the heat-affected region 308 compared to the pilot trace 320. If the speed of sound is faster in the heat-affected region 308, the signals along the beam for that particular channel will arrive earlier than expected. If the speed of sound is slower in the heat-affected region 308, the signals along the beam for that particular channel will arrive later than expected. The processor 116 calculates all of the estimated delays based on the pilot trace 320. All of the channels associated with beams having a different path length through the heat-affected region 308 will show at least some delay error when compared to the expected delays calculated from the pilot trace.

As discussed above, the path length through the heat-affected region 308 is different depending on the angle between the element/s in the array 302 and the pilot trace 320. For example, in FIG. 4, the pilot trace 320 passes through a distance 324 (also indicated by T_{P}) of the heat-affected region 308, while the beam 322 passes through a distance 326 (also indicated by T₇) of the heat-affected region 308. The distance 326 is longer than the distance 324. The path length of the beam 322 through the heat-affected region 308 is equal to 1/(T_{P} cos Θ), where Θ is the angle between the beam associated with that particular channel and T_{P} is the path length of the pilot trace through the heat-affected region 308. The exact path length though the heat-affected region 308 varies based on the angle between the element or elements associated with a particular channel and the pilot trace 320. The delay error represents the difference between the expected delay and the measured delay for each of the channels.

Based on the delay errors calculated for each of the channels in the second subset of the ultrasound channel data, the processor 116 is able to determine the time offset for each channel. The time offset represents the difference in expected time to receive the signal from a particular channel and the actual time to receive the signal from the particular channel. As was previously described with respect to step 204, the processor 116 determines an estimated size of the heat-affected region 308. Determining the estimated size of the heat-affected region 308 may include determining the thickness T_{P} of the heat-affected region 308 in an exemplary embodiment.

At step 214, the processor 116 determines that the heat-affected region 308 has experienced a temperature-induced tissue change based on the delay errors and the estimated sized of the heat-affected region 308. The temperature-induced tissue change may include either a permanent temperature-induced tissue change, such as denaturing the tissue, or a reversible temperature-induced tissue change, such as altering a speed of sound in the heat-affected region due to change in temperature in the heat-affected region 308. If there are no delay errors calculated at step 212, then the processor 116 determines that there are no temperature-induced tissue changes. According to an exemplary embodiment, determining that the tissue in the heat-affected region 308 has experienced a temperature-induced tissue change may include determining an estimated temperature of the heat-affected region 308.

The method 200 will be described in accordance with an exemplary embodiment where determining that the tissue in the heat-affected region 308 has experienced a temperature-induced tissue change includes determining an estimated temperature of the heat-affected region 308.

According to an embodiment, the processor 116 uses the thickness T_{P} of the heat-affected region 308 and the delay errors for each of the channels to calculate an estimated temperature of the heat-affected region 308. Based on the thickness T_{P} of the heat-affected region 308 and the delay errors, the processor 116 calculates what would be the required speed of sound within the heat-affected region 308 in order to account for the measured delay errors. Since the estimated size of the heat-affected region is known, such as, for instance, the thickness T_{P}, the processor 116 determines the required speed of sound for the heat-affected region 308 that would result in smaller delay errors for each ultrasound channel with respect to the pilot trace. According to one embodiment, the processor 116 may identify the speed of sound for the heat-affected region 308 that results in the smallest delay errors for the ultrasound channel data with respect to the pilot trace 320. This may be calculated, for example, by summing the absolute values of the delay error for each channel according to an embodiment. Other mathematical methods minimizing the delay errors may be used according to other embodiments.

After determining the required speed of sound, the processor 116 may determine the estimated temperature for the heat-affected region 308 that would result in the required speed of sound. According to an embodiment, the processor 116 may assume that all the tissue that is not in the heat-affected region is 37° C., which is a standard value for a human body. The processor 116 may rely on a heuristic approach or a model in order to determine the estimated temperature that would result in the required speed of sound. Additionally, according to other embodiments, both the thickness T_{P} and the estimated temperature may be determined in parallel. For example, a system of two or more models and/or equations may be solved at the same time to determine both thickness T_{P} and speed-of-sound (which is used to calculate the estimated temperature). According to other embodiments, values for both the thickness T_{P} and the estimated temperature may be iteratively calculated as one or both variables evolve over time due to the application of a thermal source.

Some embodiments may include one or more additional steps not illustrated on the flow chart shown in FIG. 2. For example, processor 116 may also use ultrasound channel data acquired from different depths in order to determine the location of the heat-affected region 308. During dynamic focusing, the processor 116 controls the focusing of the receive beams at different depths with respect to the transducer array 302. For example, the processor 116 may start by focusing at a relatively deep depth, and then acquire ultrasound channel data by focusing on a plurality of increasingly shallow depths. Dynamic receive focusing is well-known by those skilled in the art and will not be described in additional detail.

A full set of ultrasound channel data may be acquired at each depth during dynamic receive focusing. Referring to FIG. 3, the ultrasound channel data acquired while the receive focusing is at depths between the transducer array 302 and the heat-affected region 310 will not exhibit significant delay errors with respect to the estimated delay errors calculated from the pilot trace 320 since the receive focusing is a depths that are shallower than the heat-affected region 308. In contrast, there will be significant delay errors between the estimated delay and the measured delay when focusing at depths within or deeper than the heat-affected region 308 since the speed of sound is different in the heat-affected region 308 compared to the surrounding tissue.

After performing the dynamic receive focusing for a plurality of different depths, the processor 116 has ultrasound channel data associated with each of the different depths for which the dynamic receive focusing was performed. The processor 116 may then determine the approximate depth, based on the ultrasound channel data acquired from all the different focusing depths, where the delay errors start occurring. If all the data acquired from below a certain depth contains a delay error, the processor 116 may estimate that the heat-affected region 308 starts at a depth where the delay errors are first present in the ultrasound channel data. The processor 116 previously estimated the size of the heat-affected region 308 during step 204 of the method 200. Based on the previously estimated size of the heat-affected region 308 and the depth where the ultrasound channel data starts exhibiting delay errors, the processor 116 is able to calculate the position of the heat-affected region 308.

At step 216, the processor 116 presents information indicating that the heat-affected region has experienced the temperature-induced tissue change. The information may be presented in different ways according to various embodiments. For example, the processor 116 may display the estimated temperature of the heat-affected region 308. For example, the processor 116 may display one or more numbers representing temperatures within the heat-affected region 308, or the processor 116 may display a color overlay where one or more colors used in the color overlay are used to represent the estimated temperature or temperatures in the heat-affected region.

The processor 116 may present at least one of the position of the heat-affected region 308 and the estimated temperature within the heat-affected region 308. The processor 116 may, for instance, present the position of the heat-affected region 308 in a variety of different ways. The processor 116 may display a representation of the heat-affected region in the proper position with respect to an image generated from the ultrasound channel data. The representation of the heat-affected region may be positioned on either a still image generated from the ultrasound channel data, or the representation of the heat-affected region may be positioned on a live or dynamic image that is generated from the ultrasound channel data. The representation of the heat-affected region 308 may include a graphic representing the shape of the heat-affected region 308 or the representation may include the use of color to clearly demark the position of the heat-affected region 308 on the image.

The processor 116 may numerically present information regarding the position of the heat-affected region 308. For example, the processor 116 may present one or more different numbers indicating the depth, thickness, or any other attributes that would help a user to understand the position of the heat-affected region 308 with respect to one or more of the image, the probe or the patient.

FIG. 5 is a schematic representation of a display screen 500 in accordance with an exemplary embodiment. The display screen 500 represents an exemplary way that the processor 116 may present information regarding the position and temperature of the heat-affected region 308. The display screen 500 includes a B-mode image 502 and a representation of the heat-affected region 504. According to an embodiment, just the representation of the heat-affected region 504 may be shown with a color overlay and the rest of the tissue may be represented as a normal grey-scale B-mode image. The color is indicated by the hatched region within the representation of the heat-affected region 504. It should be appreciated that the color of the representation of the heat-affected region 504 may change as the temperature of the heat-affected region changes. According to another embodiment, the color overlay may cover the whole image. The colors in the color overlay may indicate the temperature of the tissue. For example, the hue of the color may be used to represent the temperature. Colors like yellow and red may be used to indicate areas of warm temperatures, while colors like blue, green, and purple may be used to indicate areas of cooler temperatures. Other embodiments may different colors to indicate temperatures.

According to another embodiment, the color of the overlay may be used to indicate when the temperature has reached a target temperature. For example, to successfully ablate tissue, the temperature must reach at least 42° C. The representation of the heat-affected region 504 may be shown in a first color, such as red, when the heat-affected region 308 has not reached the desired temperature. And, the representation of the color overlay 504 may be shown in a second color, such as green, when the temperature of the heat-affected region 308 has reached the desired temperature. It should be understood that the heat-affected region 308 may not be a uniform temperature. According to embodiments with enough ultrasound channel data, the processor 116 may represent the temperature of the heat-affected region 308 by including a color overlay of multiple different colors on the representation of the heat-affected region 504.

Providing the user with real-time feedback about the temperature and/or position of a heat-affected region provides numerous advantages. Embodiments that show the position of the heat-affected region 308 provide the user with important information regarding the location of the tissue currently being heated or cooled through an ablation procedure. By showing the location of the heat-affected region 308 on an ultrasound image, the clinician obtains real-time feedback about the position of the heat-affected region 308 with respect to a patient's anatomy. This allows the clinician to adjust the position of the ablation catheter if necessary and to monitor the position of the heat-affected region 308 during the entire ablation procedure to ensure the intended tissue is targeted by the procedure. By providing real-time feedback about temperature, various embodiments provide clinicians with real-time feedback that may be relied upon to ensure an appropriate ablation. Clinicians can adjust the power delivered to the ablation catheter and/or the rate that the ablation catheter is moved to ensure that the tissue is thoroughly ablated. Additionally, if the temperatures are too high, clinicians can decrease the power to the ablation catheter and/or move the catheter more quickly in order to minimize the risk of damaging health tissue adjacent to the intended ablation target. Embodiments that provide a warning if temperatures are too high provide a redundant patient safety feature that helps to minimize risk to the patient. Providing real-time temperature estimations based on ultrasound channel data and real-time information about the position of a heat-affected region provides clinicians with information to provide safer and more clinically effective thermal ablations. Other embodiments may simply provide notification to the user when the desired clinical outcome related to temperature-induced tissue change has been achieved.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of ultrasound imaging comprising:
acquiring ultrasound channel data for a region-of-interest;
determining an estimated size of a heat-affected region in response to an application of a thermal source within the region-of-interest;
identifying a first subset of the ultrasound channel data;
identifying a second subset of the ultrasound channel data that is different than the first subset;
generating a pilot trace based on the first subset of the ultrasound channel data;
comparing the second subset of the ultrasound channel data to the pilot trace to determine delay errors for the second subset of ultrasound channel data;
determining that the heat-affected region has experienced a temperature-induced tissue change based on the delay errors and the estimated size of the heat-affected region; and
presenting information indicating that the heat-affected region has experienced the temperature-induced tissue change after determining that the heat-affected region has experienced the temperature-induced tissue change.

2. The method of claim 1, wherein determining that the heat-affected region has experienced the temperature-induced tissue change comprises determining an estimated temperature of the heat-affected region.

3. The method of claim 1, wherein presenting the information comprises presenting the estimated temperature.

4. The method of claim 3, wherein presenting the estimated temperature comprises displaying a color overlay on a B-mode ultrasound image, where the color overlay includes a color to represent the estimated temperature.

5. The method of claim 3, further comprising determining that the estimated temperature exceeds a threshold, and wherein presenting the estimated temperature comprises providing a warning that the estimated temperature exceeds the threshold.

6. The method of claim 1, further comprising using the information indicating that the heat-affected region has experienced the temperature-induced tissue change to monitor a progress of an ablation procedure.

7. The method of claim 1, further comprising calculating a position of the heat-affected region based on the delay errors and the estimated size of the heat-affected region.

8. The method of claim 1, wherein determining the estimated size of the heat-affected region comprises implementing a heuristic approach.

9. The method of claim 8, wherein implementing the heuristic approach comprises accessing a look-up table to determine the estimated size of the heat-affected region in response to an application of the thermal source for a known amount of time.

10. The method of claim 1, wherein determining the estimated size of the heat-affected region comprises implementing a bio-heat equation.

11. The method of claim 10, wherein the bio-heat equation comprises a Pennes bio-heat equation.

12. An ultrasound imaging system comprising:
a probe;
a display device; and
a processor in electronic communication with the probe and the display device, wherein the processor is configured to:
control the probe to acquire ultrasound channel data for a region-of-interest;
determine an estimated size of a heat-affected region in response to an application of a thermal source within the region-of-interest;
identify a first subset of the ultrasound channel data;
identify a second subset of the ultrasound channel data that is different than the first subset;
generate a pilot trace based on the first subset of the ultrasound channel data;
compare the second subset of the ultrasound channel data with the pilot trace to determine delay errors for the second subset of the ultrasound channel data;
determine that the heat-affected region has experienced a temperature-induced tissue change based on the delay errors and the estimated size of the heat-affected region; and
present information indicating that the heat-affected region has experienced the temperature-induced tissue change.

13. The ultrasound imaging system of claim 12, wherein the processor is configured to determine that the heat-affected region has experienced the permanent temperature-induced tissue change by calculating an estimated temperature of the heat-affected region.

14. The ultrasound imaging system of claim 12, wherein the processor is configured to present the information by displaying the estimated temperature on the display device.

15. The ultrasound imaging system of claim 14, wherein the processor is configured to present the estimated temperature by displaying a color overlay on a B-mode image on the display device, where the color overlay includes a color representing the estimated temperature.

16. The ultrasound imaging system of claim 12, wherein the processor is configured to determine the estimated size of the heat-affected region by implementing a heuristic approach.

17. The ultrasound imaging system of claim 12, wherein the processor is configured to determine the estimated size of the heat-affected region by implementing a bio-heat equation.

18. The ultrasound imaging system of claim 12, wherein the processor is configured to receive information from an ablation system indicating a wattage and a duration of an ablation procedure, and wherein the processor is configured to use the wattage and the duration of the ablation procedure to determine the estimated size of the heat-affected region.

19. The ultrasound imaging system of claim 12, wherein the processor is configured to calculate a position of the heat-affected region based on the delay errors and the estimated size of the heat-affected region.

20. The ultrasound imaging system of claim 19, wherein the processor is configured to present information on the display device indicating the position of the heat-affected region.
